(19) United States
(12) Patent Application Publication
(10) Pub. No.: US 2015/0045884 A1
(43) Pub. Date: Feb. 12, 2015

(54) STAPES PROSTHESIS WITH SNAP CLOSURE

(71) Applicant: HEINZ KURZ GMBH MEDIZINTECHNIK, DUSSLINGEN (DE)

(72) Inventors: ALBRECHT EIBER, WEINSTADT (DE); UWE STEINHARDT, HIRRLlNGEN (DE); HEINZ KURZ, DUSSLINGEN (DE)

(21) Appl. No.: 14/453,836

(22) Filed: Aug. 7, 2014

(30) Foreign Application Priority Data
Aug. 8, 2013 (DE) 102013108566.0

(51) Int. Cl. A61F 2/18

(52) U.S. Cl. CPC A61F 2/18; A61F 2002/183
USPC 623/10

(57) ABSTRACT

An ossicular prosthesis includes a sound-conducting prosthesis body with a first coupling element designed for the mechanical connection to the limb of incus (A) at one end and a second coupling element for the mechanical connection to parts of a component of the ossicular chain or directly to the inner ear, The first coupling element has a receiving part rigidly connected to the prosthesis body to accommodate the free end section of the limb of incus, including a U-shaped cliplock rotatably supported in the receiving part and swivellable over the free end section of the limb of incus in order to secure the limb of incus. The cliplock has at least one bulge directed toward the receiving part that protrudes from the cliplock so far that said bulge touches an outer surface of the receiving part when swivelled.

### CROSS-REFERENCE TO A RELATED APPLICATION

The invention described and claimed hereinbelow is also described in German Patent Application DE 10 2013 108566.0, filed on Aug. 8, 2013. The German Patent Application, the subject matters of which is incorporated herein by reference, provides the basis for a claim of priority of invention under 35 U.S.C. 119(a)-(d).

### BACKGROUND OF THE INVENTION

The present invention relates to an ossicular prosthesis used to replace or bridge at least one element of the human ossicular chain, which comprises an elongated, sound-conducting prosthesis body with a first coupling element having a receiving part designed for mechanical connection to a limb of incus, in particular, the free end section of the limb of incus, the first coupling element further having a cliplock that is curved to be substantially U-shaped, the cliplock rotatably supported in the receiving part and swivellable over the free end section of the limb of incus to secure the limb of incus, the prosthesis body including on its other end a second coupling element for the mechanical connection to a further component or parts of a component of the ossicular chain or directly to the inner ear.

Passive ossicular prostheses, which are well known, are usually designed as stapes prostheses. Such ossicular prostheses described, for example, in U.S. Pat. No. 3,196,462, U.S. Pat. No. 4,292,693 and US 2006/0241755 A1, to name just a few. In the ossicular prosthesis according to U.S. Pat. No. 3,196,462, the cliplock is not curved so as to be U-shaped, but rather to be O-shaped. Stapes prostheses that are similar, but do not have the handle-shaped design of the cliplock are known from U.S. Pat. No. 3,711,869 and U.S. Pat. No. 3,931,648.

The human middle ear comprising the ossicles thereof transmits sound waves impacting the tympanic membrane via the external auditory meatus to the inner ear, which is filled with fluid. The three ossicles are the hammer (lat. malleus), which is fastened to the tympanic membrane, the stirrup (lat. stapes), which is connected via the footplate (lat. basis stapedis) thereof to the inner ear, and the anvil (lat. incus), which is located between the hammer and the stapes and is hingedly connected thereto.

Otosclerosis is a disease of the human petrosal bone (=bone in which the entire ear is seated), in which inflammation-like bone remodeling processes can result in fixation of the stapes, which normally swings freely. As a result, the sound signal is transmitted not at all or incompletely via the ossicular chain to the inner ear, thereby resulting in hearing loss.

Ossicular prostheses are used to improve sound transmission in patients having different pathologies, for example, to conduct sound from the tympanic membrane to the inner ear in cases in which the ossicles of the human middle ear are missing or damaged, either entirely or partially. The ossicular prosthesis has two ends. Depending on the specific circumstances, one end of the ossicular prosthesis is fastened to the tympanic membrane or an ossicles, and the other end of the ossicular prosthesis is fastened to the stapes of the human ossicular chain or is inserted directly into the inner ear.

Three types of ossicular prostheses that are used particularly frequently are stapes prostheses, partial prostheses and total prostheses.

Stapes prostheses are usually fixed on the long limb of incus and extend via a piston into the inner ear or are seated, with the piston, on a piece of tissue that seals the inner ear. Partial prostheses typically bear via a top plate against the tympanic membrane and establish a connection to the head of the stapes. Total prostheses connect the tympanic membrane to the base of stapes.

In addition, a distinction is generally made between passive ossicular prostheses and active hearing implants having electronic amplification elements, which are operated via an external energy part. The initially defined ossicular prosthesis of the type in question is a passive prosthesis, in which a receiving part is provided on the upper end of the prosthesis body and can be placed under the free end section of the limb of incus. In order to secure the prosthesis on the incus, a movable cliplock curved so as to be U-shaped is then swivelled over the limb of incus.

In all such ossicular prostheses described in the aforementioned prior art (with the exception of the prosthesis according to U.S. Pat. No. 4,292,693), however, none comprise a safeguard against the cliplock inadvertently sliding off on its own after having been secured, by the surgeon, in the patient's middle ear. The clip can therefore swivel back, on its own, over the free end section of the limb of incus immediately after conclusion of implantation. One reason that this might occur is as a result of postsurgical cicatricial pulls during the healing process. Subsequently thereto, it is likely that the placement of the limb of incus on the receiving part also will slip and the ossicular prosthesis will then “dangle freely in the air”, interrupting the sound-conducting connection to the inner ear and ruining the success of the surgery on the middle ear that was just completed.

The ossicular prosthesis according to U.S. Pat. No. 4,292,693 attempts to overcome this considerable disadvantage by applying two opposing cams on the cylindrical outer wall of the receiving part, which is rigidly connected to the prosthesis body. The cams have slanted outer surfaces that extend toward one another at an angle and terminate in a shoulder, which abruptly extends in the direction toward the receiving part. This design is intended to enable the lever-shaped cliplock to be drawn over the rising outer surfaces of the cams and, thereby, over the free end section of the limb of incus during closure thereof, but to subsequently prevent the cliplock from swivelling back on its own.

The disadvantage of this known ossicular prosthesis, however, is the relatively complex design. That is, to properly utilize same, the practitioner must understand the tiny dimensions within which such cams on the receiving part will have to move. The receiving part itself typically has a diameter in the range between one-half and one millimeter. The cams, which have a relatively complicated design, must be manufactured in a magnitude of 100 μm and less, which is a nearly unattainable challenge in terms of production engineering, even in the current age of laser technology (that is, over three decades after publication of U.S. Pat. No. 4, 292, 693). Needless to say that it is not feasible to manufacture such cams in notable quantities and at a competitive price.

This is likely the reason why the ossicular prostheses according to U.S. Pat. No. 4,292,693 is nota commercially successful to date, which also is implied, for example, from US Pub. Appln. Ser. No. 2006/0241755 A1, which extensively describes and references the prior art according to U.S. Pat. No. 4,292,693, including the above-described retaining function using the cams, although this is not at all considered for the invention disclosed in US Pub. Appln. Ser. No. 2006/0241755 itself.

A further serious disadvantage of the “cam solution” is the risk of the cliplock tearing off while being drawn over the cams. That is, if the cams are disposed with the slanted outer surfaces thereof rotated even by a small angle against the pulling direction of the clip, which can easily occur in production engineering due to the finite tolerances, or if the cams protrude slightly too far out of the receiving part, in many cases the clip will become hung up on the cam and simply become torn apart as the movement continues. The negative consequences can be devastating to the patient, in particular if the process goes unnoticed during implantation. Due to the relatively tight spaces and the fact that the view is not always clear, this can result in the surgeon not recognizing the problem, continuing to attempt to draw the clip over the limb of incus and, thereby irreparably damaging the inner ear by the implant plunging therein in an uncontrolled manner.

### SUMMARY OF THE INVENTION

The present invention overcomes the shortcomings of known arts, such as those mentioned above.

To that end, the present invention provides a passive ossicular prosthesis using the simplest technical means possible and cost-effectively such that the inventive ossicular prosthesis is produceable particularly easily and economically in terms of production engineering. In addition, the inventive ossicular prosthesis reliably avoids the above-described risks associated with the use of an ossicular prosthesis according to the aforementioned U.S. Pat. No. 4,292,693, for example, wherein the ossicular prosthesis still offers good protection against the cliplock inadvertently retracting over the limb of incus after completion of implantation.

According to the invention, the cliplock of the initially described ossicular prosthesis is modified to include at least one bulge, which is directed toward the receiving part and protrudes from the cliplock so far that the bulge touches an outer surface of the receiving part when the cliplock is swivelled.

In this manner it is possible to reliably prevent the situation in which the cliplock, after having been swivelled over the free end section of the limb of incus, swivels back on its own and thereby releases the connection of the ossicular prosthesis from the limb of incus. In addition, there is no risk that the cliplock will become damaged during implantation or thereafter. Finally, the design according to the invention is substantially less complicated and, therefore, economical when considered in view of the above entioned prior art, such as U.S. Pat. No. 4,292,693.

Due to the clearly definable bulge on the wire and the defined diameter at the receiving part, and due to the clearly defined geometry of the U-part, the force to be applied in order to elastically open the U-part is clearly predictable. The inventive system therefore becomes much more reliable than the other ossicular prosthetic system in terms of application.

Furthermore, the geometry and dimensions of the clip can be designed such that slight pressure on the limb of incus is always present, which has the advantage that there is no “play” at the clip. If one considers the risk of the prosthesis inadvertently plunging into the inner ear, it quickly becomes clear that this is prevented by the clip and the self-retaining force thereof.

In an embodiment of the ossicular prosthesis according to the invention, the bulge protrudes from the cliplock so far that, during the swivelling of the cliplock, the bulge bears at least temporarily with pressure against the outer side of the receiving part or slides thereon with pressure and the cliplock is made of a material that elastically deforms during swivelling due to the effect of the pressure. The force prevents the swivelling back and provides the implant with a safeguard against slipping off or plunging.

A class of embodiments of the invention that is advantageous in terms of handling and that is particularly cost-effective to manufacture is characterized in that the cliplock is formed of wire-shaped material, and the bulge has the shape of a bead and protrudes in the direction toward the receiving part at least by the distance of one-fourth of the wire thickness, preferably by one-half of the wire thickness. This type of manufacture makes it possible to exactly define the force and dimensions, in order to ensure clear reproducibility.

Preferably, the wire-shaped material with which the cliplock is formed has a round cross section. This type of production makes it possible to obtain very cost-effective variants of the invention. In alternative embodiments, the wire-shaped material of which the cliplock is made has a rectangular, preferably square cross section. In particular, the cliplock can be made of strip-shaped material.

In an embodiment, the ossicular prosthesis according to the invention includes that the U-shaped cliplock comprises two side sections, which extend substantially parallel and are connected to one another at one end via a clip section and, at the other end, each transition into end pegs, which rotatably engage into the receiving part, and in that at least one bulge is disposed on each of the two side sections. As a result, the acting force can be set quasi via the geometry.

This embodiment can be further improved by bending the U-shape of the cliplock in the region of the clip section out of the plane formed by the U-shape, which has the advantage that the deflection is partially shortened, approximately, laterally toward the tympanic membrane, thereby simplifying the implantation in terms of the overall size.

In an embodiment, the ossicular prosthesis according to the invention is characterized in that the prosthesis body comprises at least one joint, in particular a ball joint. This is advantageous in terms of particularly high postsurgical mobility of the prosthesis. Developments are also possible in which a plurality of mutually adjacent, further rotary elements is provided, preferably a ball joint chain. Once the prosthesis has been surgically implanted in the middle ear and the tympanic membrane has been closed, the recovery phase begins. Scars form during this period, and they produce unforeseeable forces which can cause the prosthesis to move out of localized position thereof.

The ossicular prosthesis, according to the invention, or parts thereof may be made of titanium and/or gold and/or tantalum and/or steel, and/or an alloy of said metals. It is known that titanium, in particular, in addition to being stiff and having excellent sound-conducting properties, also exhibits excellent biocompatibility with the human ear.

In terms of the postsurgical position adjustment described above, embodiments of the invention are advantageous in which the ossicular prosthesis or parts thereof are composed of a material having memory effect or superelastic properties, for example, Nitinol in particular, as is known per se, for example, from WO 02/069850 A1 or U.S. Pat. No. 6,554,861.

Ossicular prostheses according to the present invention also may be composed of a ceramic material. Also, the entire prosthesis or parts thereof may be made of biocompatible plastics, particularly silicone, polytetrafluoroethylene (PTFE) or fibrous composite materials. With these materials, postsurgical rejection reactions also may be prevented in most cases.

In an embodiment, the ossicular prosthesis of the present invention is constructed so a mass distribution of the individual parts of the prosthesis is calculated depending on a desired, specifiable frequency response of sound conduction in the middle ear. This allows the sound propagation properties to be tuned to a certain extent using a custom-made ossicular prosthesis without a great deal of additional technical complexity. The specific frequency adaptation for improved sound conduction in the middle ear is described, for example, in EP 1 706 071 and U.S. Pat. No. 7,871,439.

In special embodiments, such “mechanical tuning” may be achieved, for example, by fastening at least one additional mass to a part of the ossicular chain or the prosthesis depending on a desired, specifiable frequency response of sound conduction in the middle ear. The additional mass is fastened to a part of the ossicular chain or the prosthesis using a clip. The additional mass and/or clip may be coated with a biologically active coating.

### BRIEF DESCRIPTION OF THE DRAWINGS

Further features and advantages of the invention will become apparent from the description of embodiments that follows, with reference to the attached figures, wherein:

FIG. 1 is a schematic spatial representation of an ossicular prosthesis according to the invention after implantation, highlighting a cliplock made of round wire and swivelled over the free end section of the limb of incus and including a second coupling element configured as a piston for engagement, through a perforated base of stapes, into the inner ear;

FIG. 2 is a schematic spatial representation of an ossicular prosthesis similar to that depicted in FIG. 1;

FIG. 3 is a schematic spatial representation of an ossicular prosthesis depicted in FIG. 2, with a cliplock made of wire having a square cross-section and having bores in the clip and a bulge that protrudes only slightly inwardly and is directed toward the receiving part;

FIG. 4 is a schematic spatial representation of an ossicular prosthesis depicted in FIG. 3, without bores in the clip but including a bulge configured in the shape of beading;

FIG. 5 is a schematic spatial representation of an ossicular prosthesis depicted in FIG. 4, but with clearly deeper beading, which protrudes approximately by the distance of one-half of the wire thickness in the direction toward the receiving part; and

FIG. 6 is a schematic spatial representation of an ossicular prosthesis depicted in FIG. 1, comprising a second fastening element placed onto an artificial base of stapes.

### DETAILED DESCRIPTION OF THE PREFERRED EMBODIMENTS

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

Embodiments of the ossicular prosthesis 10; 20; 30; 40; 50; 60 according to the invention are schematically depicted in the figures in different levels of detail. Each invention ossicular prostheses embodiment comprises a shank-shaped, elongated, sound-conducting prosthesis body 13; 23; 33; 43; 53; 63, each of which comprises, at one end, a first coupling element 11; 21; 31; 41; 51; 61, which is designed for the mechanical connection to the limb of incus A, and a receiving part 11a; 21a; 31a; 41a; 51a; 61a, which is rigidly connected to the prosthesis body 13; 23; 33; 43; 53; 63, for accommodating the free end section of the limb of incus A, and each of which comprises a cliplock 11b; 21b; 31b; 41b; 51b; 61b, which is curved so as to be substantially U-shaped, is rotatably supported in the receiving part 11a; 21a; 31a; 41a; 51a; 61a, and can be swivelled over the free end section of the limb of incus A accommodated in the receiving part 11a; 21a; 31a; 41a; 51a; 61a in order to secure the limb of incus A. Attached at the other end of the prosthesis body 13; 23; 33; 43; 53; 63 is a second coupling element 12; 22; 32; 42; 52; 62, which can be configured in diverse geometrical shapes for the mechanical connection to a further component or parts of a component of the ossicular chain, or can be configured as a piston for direct insertion into the inner ear.

According to the invention, the ossicular prosthesis 10; 20; 30; 40; 50; 60 is characterized in that the cliplock 11b; 21b; 31b; 41b; 51b; 61b comprises at least one bulge 111; 211; 311; 411; 511; 611′, 611″, which is directed toward the receiving part 11a; 21a; 31a; 41a; 51a; 61a and protrudes from the cliplock 11b; 21b; 31b; 41b; 51b; 61b so far that the bulge touches an outer surface of the receiving part 11a; 21a; 31a; 41a; 51a; 61a when the cliplock 11b; 21b; 31b; 41b; 51b; 61b is swivelled.

The bulge 111; 211; 311; 411; 511; 611′, 611″ protrudes from the cliplock 11b; 21b; 31b; 41b; 51b; 61b so far that, during the swivelling of the cliplock 11b; 21b; 31b; 41b; 51b; 61b, said bulge bears at least temporarily with pressure against the outer side of the receiving part 11a; 21a; 31a; 41a; 51a; 61a or slides thereon with pressure, and the cliplock 11b; 21b; 31b; 41b; 51b; 61b is made of a material that elastically deforms during swivelling due to the effect of the pressure.

The cliplock 11b; 21b; 31b; 41b; 51b; 61b is formed of wire-shaped material, wherein the bulge 111; 211; 311; 411; 511; 611′, 611″ has the shape of a bead and protrudes in the direction toward the receiving part 11a; 21a; 31a; 41a; 51a; 61a at least by the distance of one-fourth of the wire thickness, preferably by one-half of the wire thickness.

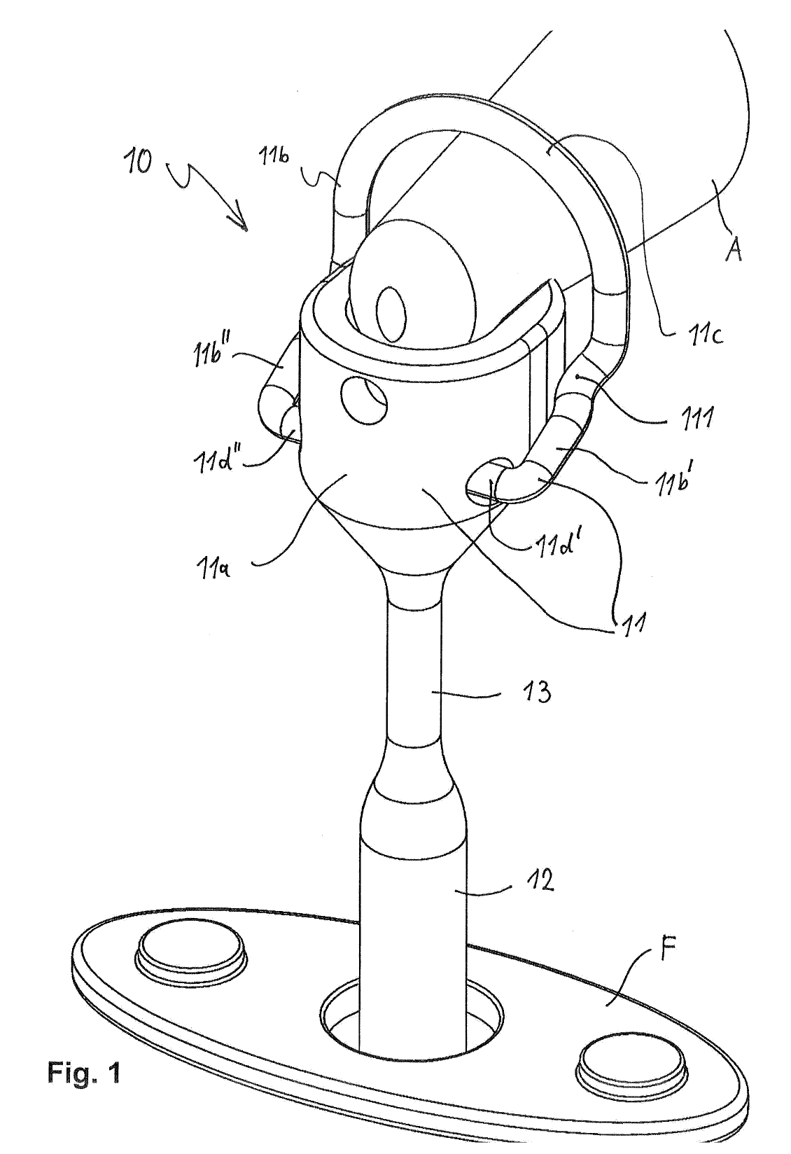

In FIG. 1, a second coupling element 12, which is configured as a piston, engages through a perforated base of stapes F into the inner ear. The base of stapes is depicted in a stylized manner having having both attachments of the previous stapes.

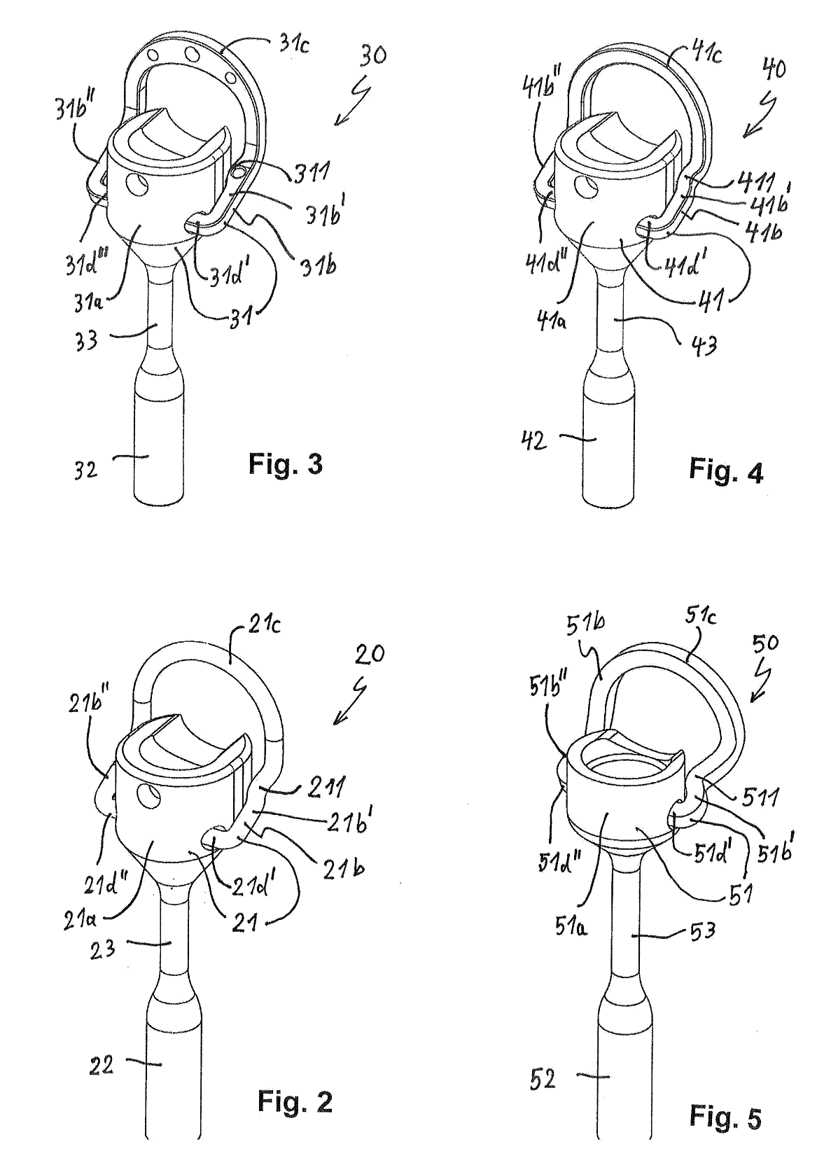

In the embodiments of the ossicular prosthesis 10; 20; 60 according to the invention shown in FIGS. 1, 2 and 6, the wire-shaped material of which the cliplock 11b; 21b; 61b is made has a round cross-section. In the embodiments according to FIGS. 3 to 5, the wire of the cliplock 31b; 41b; 51b has a rectangular, preferably square cross-section.

The U-shaped cliplock 11b; 21b; 31b; 41b; 51b; 61b preferably comprises two side sections 11b′, 11h″; 21b′, 21b″; 31b′, 31b″; 41b′, 41b″; 51b′, 51b″; 61b′, 61b″, which extend substantially parallel and are connected to one another at one end via a clip section 11c; 21c; 31c; 41c; 51c; 61c and, at the other end, each transition into end pegs 11d′, 11d″; 21d′, 21c1″; 31d′, 31d″; 41d′, 41d″; 51d′, 51d″; 61d′, 61c1″, which rotatably engage into the receiving part 11a; 21a; 31a; 41a; 51a; 61a.

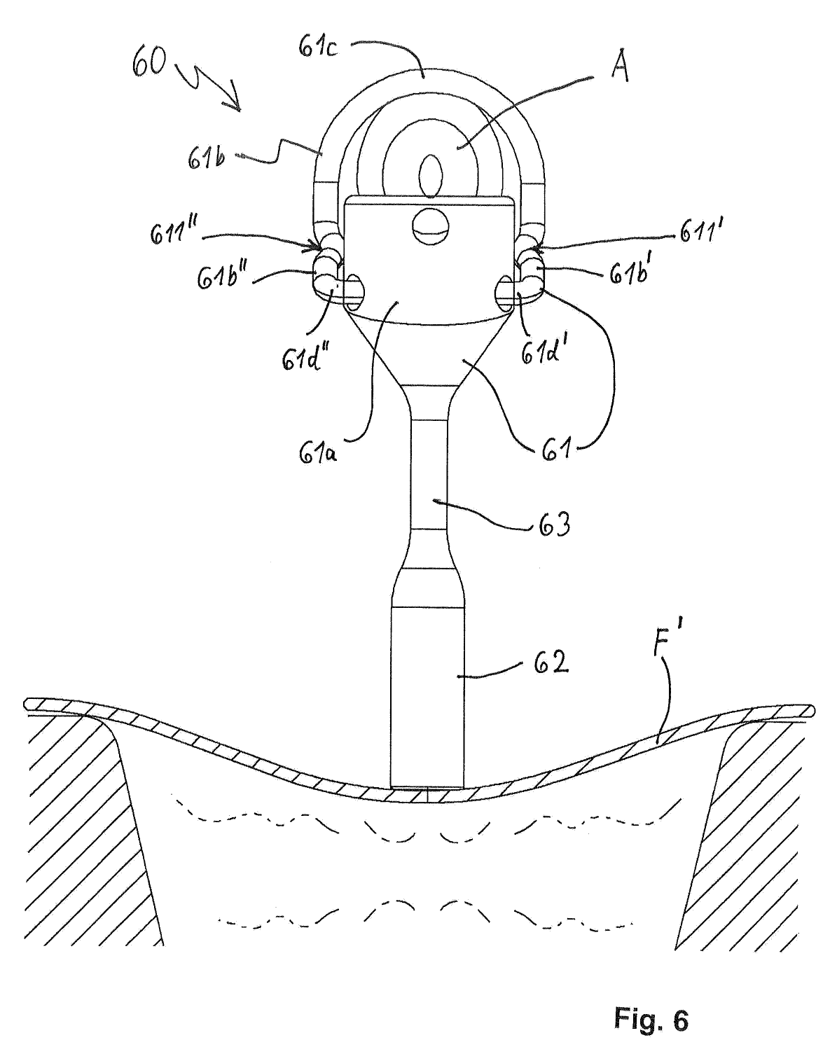

As is evident from FIG. 6 in particular, at least one bulge 611′, 611″ can be disposed on both side sections 61b′, 61b″, respectively. In this case, the second coupling element 62 is usually seated on a base of stapes P that is artificially produced, generally from tissue of the patient.

The U-shape of the cliplock 11b; 21b; 31b; 41b; 51b; 61b is preferably bent in the region of the clip section 11c; 21c; 31c; 41c; 51c; 61c out of the plane formed by the two limbs of the U-shape.

In embodiments of the invention that are not depicted in the drawing, the prosthesis body can comprise at least one joint, in particular a ball joint.

A mass distribution of the individual parts of an ossicular prosthesis according to the invention can be calculated depending on a desired, specifiable frequency response of sound conduction in the middle ear such that it is possible to tune the sound propagation properties in an individualized manner. This also can be achieved by way of trimming masses that can be clipped to the ossicular prosthesis in embodiments of the invention that are not depicted in the drawings.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. A ossicular prosthesis for replacing or bridging at least one element of the human ossicular chain, comprising:
an elongated, sound-conducting prosthesis body;
a first coupling element rigidly connected at one end of the elongated, sound-conducting prosthesis body, the first coupling element including a receiving part for accommodating and mechanically connecting to a free end section of the limb of incus and a cliplock, curved to be substantially U-shaped, rotatably supported in the receiving part and configured to be swivelled over the free end section of the limb of incus accommodated in the receiving part in order to secure the limb of incus; and
a second coupling element connected at the other end of the elongated, sound-conducting prosthesis body for mechanical connecting to a further component or parts of a component of the ossicular chain or directly to the inner ear;
wherein the cliplock has at least one bulge directed toward the receiving part and protruding from the cliplock so far that the bulge touches an outer surface of the receiving part when the cliplock is swivelled.

2. The ossicular prosthesis according to claim 1, wherein the bulge protrudes from the cliplock so far that, during the swivelling of the cliplock, the bulge bears at least temporarily with pressure against the outer side of the receiving part or slides against the outer side of the receiving part with pressure, and wherein the cliplock is made of a material that elastically deforms during swivelling due to the effect of the pressure.

3. The ossicular prosthesis according to claim 1, wherein the cliplock is formed of wire-shaped material and wherein the bulge is shaped as a bead and protrudes in a direction toward the receiving part at least by the distance equal to one-fourth of the wire thickness.

4. The ossicular prosthesis according to claim 3, wherein the bulge protrudes in the direction toward the receiving part at least by the distance equal to one-half of the wire thickness.

5. The ossicular prosthesis according to claim 3, wherein the wire-shaped material of which the cliplock is formed has a round cross-section.

6. The ossicular prosthesis according to claim 3, wherein the wire-shaped material of which the cliplock is formed has a rectangular cross-section.

7. The ossicular prosthesis according to claim 1, wherein the U-shaped cliplock comprises two side sections that extend substantially in parallel and that are connected to one another at one end via a clip section and, at the other end, each of the side sections transition into end pegs, which rotatably engage into the receiving part and wherein at least one bulge is disposed on each of the two side sections.

8. The ossicular prosthesis according to claim 7, wherein the U-shape of the cliplock is bent in the region of the clip section out of the plane formed by the U-shape.

9. The ossicular prosthesis according to claim 1, wherein the ossicular prosthesis is composed entirely or partially of a material having memory effect, having superelastic properties or both.

10. The ossicular prosthesis according to claim 1, wherein the prosthesis body comprises at least one joint.
